(19) United States
(12) Patent Application Publication
(10) Pub. No.: US 2021/0228410 A1
(43) Pub. Date: Jul. 29, 2021

(54) Eye protection for Corona virus

(71) Applicants: David Byron Douglas, Winter Park, FL (US); Robert Edwin Douglas, Winter Park, FL (US)

(72) Inventors: David Byron Douglas, Winter Park, FL (US); Robert Edwin Douglas, Winter Park, FL (US)

(21) Appl. No.: 16/773,960

(22) Filed: Jan. 27, 2020

(51) Int. Cl. A61F 9/02; B01D 46/42; B01D 46/00; A42B 3/18

(52) U.S. Cl. CPC A61F 9/029; B01D 46/4272; B01D 46/0028; A61F 2009/021; A61F 9/028; B01D 2265/04; B01D 2279/40; A42B 3/18

(57) ABSTRACT

This patent discloses an apparatus that protects a user's eyes from the dangerous particulates in the atmosphere. A high efficiency filter is utilized in conjunction with goggles or eye glasses to prevent dangerous particulates in the environment from reaching the compartment of air that touches the eyeballs. This apparatus is designed to help mitigate the spread of the Corona Virus outbreak.

### TECHNICAL FIELD

Aspects of this disclosure are generally related to eye protection.

### BACKGROUND

Outbreaks of viruses can be a public health emergency. Of particular concern is the Corona Virus outbreak.

### SUMMARY

All examples, aspects and features mentioned in this document can be combined in any technically possible way.

The purpose of this invention is to improve barrier precautions through development of a low cost eye protection to prevent the spread of disease.

The preferred embodiment of this invention is an apparatus worn on a user's face. More specifically, the preferred embodiment is a device that is most similar appearing to ski goggles; however, it is equipped with a high efficiency air filter to prevent dangerous viruses (e.g., Corona virus) or particles in the air from contacting the surface of the eyeballs. The preferred embodiment provides a safe medium of air outside the eyeballs free of viruses and particles. The preferred embodiment is comfortable to wear, which will encourage good compliance. Thus, the user may wear the goggles for long periods (e.g., 16 hours) during the day time and then go home and take them off for the evening time (e.g., per user discretion). Furthermore, the preferred embodiment may include a removable filter component, such that it can be cleaned or replaced at the end of the day. The preferred embodiment includes a set of goggles, a divider, which is placed between the lenses of the goggles and the skin, and a filter placed within the divider. The divider separates an air mass that touches the user's eyes and an external air mass wherein at least some portion of the divider contains a highly effective air filter.

Some embodiments of the divider comprise wherein the entire divider is comprised of a filter, which would maximize air exchange between the inner air mass touching the eyeballs and the outer air mass from the external environment. Some embodiments of the divider comprise wherein at least one portion of the divider is impermeable to gas and at least one portion of the divider is permeable to gas (e.g., via the filter). The purpose of the air exchange is to allow for comfort over long periods of wear. Without any air exchange, it is possible that the inside of the lenses of the goggles fogs up over time. This may make it difficult for the user to see through the goggles and limit daily activities. The preferred embodiment is meant to be worn in conjunction with a high efficiency air filter mask (e.g., N95 mask). This preferred embodiment is more comfortable and has higher compliance than a set of hermetically sealed swimming goggles and N95 mask, since the swimming goggles fogged up after 5 minutes for a user.

In some embodiments, the bioactive substances can be placed in the filter, such as colloidal silver. Other substances such as activated charcoal can be placed.

Some embodiments further comprising wherein the divider forms a substantially hermetic seal to at least one of the group comprising the skin on the user's face and the goggles. Some embodiments comprise wherein the hermetic seal comprises at least one of the group of rubber, plastic, silicone, tape, metal fixation devices, non-metal fixation devices or other similar materials.

Some embodiments comprise wherein the goggles comprises at least one of the group of thin plastic surface(s), prescription eye glasses, and sunglasses. Some embodiments comprise wherein the air gap in front of the eyeballs is large enough for a user to wear his or her own glasses underneath. Some embodiments comprise wherein a hole in the divider creates an air-tight seal with the frame of the glasses, so as to prevent a tiny air gap from forming near where the user's glasses meet the side of the face.

Some embodiments comprise wherein the air filter comprises at least one of the group of an N95 filter and N99 filter. A large array of high efficiency filters could be incorporated into the overall design.

Some embodiments comprise wherein a device is used to keep the divider or seal firmly pressed against the skin of the face comprising at least one of the group of string, ties, rope, springs, magnets, or other similar type devices. The preferred embodiment is a strap extending around the top, sides and back of the user's head.

Some embodiments comprise wherein a one way valve is placed at a portion of the divider and wherein the filter is placed in proximity to the one way valve. Some embodiments comprise wherein multiple one way valves are placed. Some embodiments comprise wherein a one way valve is placed in close proximity to a filter, so that air is filtered prior to entering from the external environment into the air mass compartment touching the eyeballs. Some embodiments comprise wherein a one way valve is placed in close proximity to a filter prior to air exiting from the air mass compartment touching the eyeballs to the external environment, so that any backwards airflow (i.e., opposite of the direction of that air is supposed to flow through the one way valve) would be filtered.

Some embodiments comprise wherein the filter is a small (e.g., quarter sized) filter placed over the one way valve. Some embodiments comprise wherein the small e.g., quarter sized) filters can be removed and replaced on a daily basis.

Some embodiments comprising wherein the filter can be sterilized. Processes such as chemical sterilization (e.g., Clorox bleach) and non-chemical sterilization (e.g., light, heat, etc.). can be used for sterilization.

An alternative embodiment includes wherein an apparatus worn on a user's face comprising: a set of goggles wherein there is at least one portion that is hermetically sealed to the user's face and wherein there is at least one portion that is not hermetically sealed; and a filter placed over the at least one portion of the goggles that is not hermetically sealed.

Another alternative embodiment comprises wherein an apparatus worn on a subject's face comprises a set of goggles wherein at least one lens of the set of goggles contains holes and an air filter placed at the sites of the holes. The benefit of this embodiment would be that a standard hermetically sealed pair of goggles (e.g., a pair of swimming goggles) could be converted to a pair of non-hermetically sealed pair of goggles, which is safe to wear over long periods of time due to the fact that there is a high efficiency filter in place. In this embodiment, the air filter placed at the sites of the holes can be replaced. For example, an air filter can be manufactured in a circle and the outer circumference of the circle having a small (e.g., 0.5 cm) region of tape, such that it can be stuck onto the goggles.

The preferred embodiment is to incorporate a version of this apparatus into daily life. For example, the embodiment would ideally be comfortable enough (e.g., light weight, does not fog up, wide field of view, etc.) for 12 hours of continuous wear. Additionally, the device would need to be able to be disinfected easily (e.g., from chemical wipe down) or from heating sources (e.g. heating to temperature between 165-212 degrees Fahrenheit).

### BRIEF DESCRIPTION OF THE FIGURES

FIG. 1A illustrates the front, oblique view of an embodiment of the apparatus.

FIG. 1B illustrates a top down view of an embodiment of the apparatus.

FIG. 2A illustrates a top down view of an embodiment of this apparatus, which is customizing a user's glasses to include a filter component.

FIG. 2B illustrates a side view of an embodiment of this apparatus, which is customizing a user's glasses to include a filter component.

FIG. 2C illustrates a side view of a user wearing an embodiment of this apparatus, which is customizing a user's glasses to include a filter component.

FIG. 3A illustrates a front view of an example embodiment wherein the filters are placed on the lenses of eye glasses.

FIG. 3B illustrates a front view of an example embodiment wherein the filters are placed on the lenses of goggles.

FIG. 3C illustrates a non-adhesive side view of a small filter sized designed to fit over the lens of the goggles.

FIG. 3D illustrates an adhesive side view of a small filter sized designed to fit over the lens of the goggles.

### DETAILED DESCRIPTION

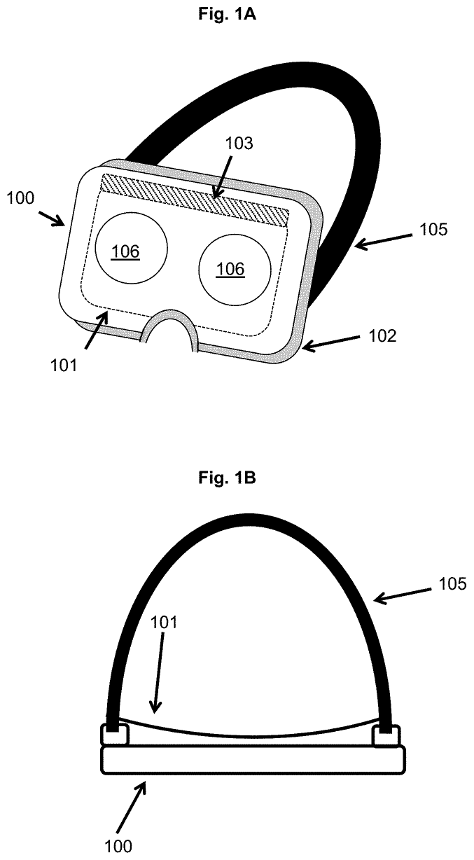

FIG. 1A illustrates the front, oblique view of an embodiment of the apparatus. 100 illustrates an external frame of the goggles. 101 illustrates an internal frame within the goggles, which provides rigidity and keeps the lenses away from the eyes. 102 illustrates a material to form an air tight (or near air tight) seal between with the face. Note that this portion could be made out of rubber, silicone or similar type materials. Some of the materials could be stretchable. It could also be air filled, so as to provide better contact with the skin, such as is performed in a bag valve mask. 103 illustrates an air filter. Note that the preferred embodiment is an N95 filter, but other types of filters could also be used. In addition, the size and position of the filter could be variable. 105 illustrates a strap to secure the goggles to the head. Other securing devices could be implemented. 106 illustrates the lenses. Various configurations and materials of the lenses could be utilized.

FIG. 1B illustrates a top down view of an embodiment of the apparatus. 100 illustrates an external frame of the goggles. 101 illustrates an internal frame within the goggles, which provides rigidity and keeps the lenses away from the eyes. 105 illustrates a strap to secure the goggles to the head.

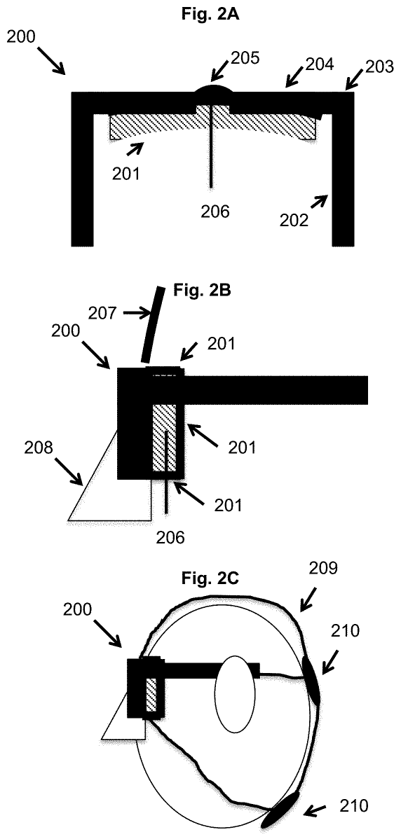

FIG. 2A illustrates a top down view of an embodiment of this apparatus, which is customizing a user's glasses to include a filter component. 200 illustrates the apparatus. 201 illustrates the portion of the apparatus that forms a substantially (approximately or near complete) hermetic seal with the user's forehead. 202 illustrates the frame, which courses over the side of the face and fits over the person's ear. 203 illustrates a hinge. Note that a hinge would be on both sides. 204 illustrates a lens. 205 illustrates the bridge of the glasses. 206 illustrates the filter. Note that the specific filter can vary in design, type (e.g., N95 or N99), size or other aspects. It could be designed to be replaceable or other design features, even cosmetic, can be implemented.

FIG. 2B illustrates a side view of an embodiment of this apparatus, which is customizing a user's glasses to include a filter component. 200 illustrates the apparatus. 201 illustrates the substantially (approximately or near complete) hermetic seal to the forehead 207 and the side of the face (e.g., lateral and posterior to the eye). 206 illustrates the filter. Note that the specific filter can vary in design, type (e.g., N95 or N99), size or other aspects. It could be designed to be replaceable or other design features, even cosmetic, can be implemented. 208 illustrates the nose.

FIG. 2C illustrates a side view of a user wearing an embodiment of this apparatus, which is customizing a user's glasses to include a filter component. 200 illustrates the apparatus. 209 illustrates straps securing the apparatus to the patient's head snugly so a substantially hermetic seal can be achieved. 210 illustrates tightening devices at the back of the head.

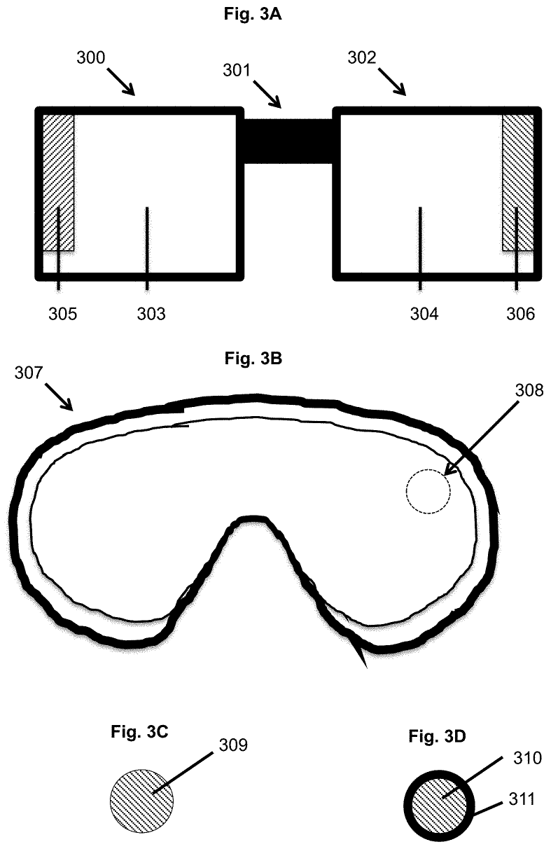

FIG. 3A illustrates a front view of an example embodiment wherein the filters are placed on the lenses of eye glasses. 300 illustrates the frame around the right lens. 301 illustrates the bridge portion of the frame. 302 illustrates the frame around the left lens. 303 illustrates the right lens. 304 illustrates the left lens. 305 illustrates a filter (e.g., N95) attached (substantially hermetically sealed) to the right lens. Note that the substantially hermetically sealed causes air mass that traverses from the external environment (e.g., in the room where the person is exposed to the Corona virus) into the air mass that touches the user's eyeball to be filtered. 306 illustrates a filter (e.g., N95) attached (substantially hermetically sealed) to the left lens. Note that the substantially hermetically sealed causes air mass that traverses from the external environment (e.g., in the room where the person is exposed to the Corona virus) into the air mass that touches the user's eyeball to be filtered.

FIG. 3B illustrates a front view of an example embodiment wherein the filters are placed on the lenses of goggles. 307 illustrates a set of goggles. 308 illustrates a hole in the set of goggles. In this embodiment, the hole is dime sized.

FIG. 3C illustrates a non-adhesive side view of a small filter sized designed to fit over the lens of the goggles. 308 illustrates the side of a small filter not touching the lens with size designed to fit over the hole 308 in the goggles 307, as shown in FIG. 3B, with enough coverage to have some overlap with the lens. For example, the size of the filter could be quarter sized.

FIG. 3D illustrates an adhesive side view of a small filter sized designed to fit over the lens of the goggles. 309 illustrates the side of a small filter touching the lens with the size designed to fit over the hole 308 in the goggles 307, as shown in FIG. 3B, with enough coverage to have some overlap with the lens. 311 illustrates a ring of adhesive. Types of adhesive could vary. For example, the size of the filter could be quarter sized. The goal of this embodiment is for the user to be able to prep the goggles (e.g., put the filter 310 and affix the adhesive portion 311 to the lens and adequately cover the hole 308), then put on the goggles at the beginning of the day and wear them all day long when out and about in the daily activities of life. Then, at the end of the day, return home, discard the adhesive filter and wash the goggles, wash their hands, etc. This unit is meant to be worn with the a separate N95 mask. This unit is designed to be far more comfortable than a full gas mask covering the mouth, face, eyes, nose, etc. This will result in greater compliance. In the event that a user decides to use the goggles rather than the modified eye glasses, as described in FIGS. 2A, 2B and 2C, the user could fit prescription eye glasses inside of the goggles.

1. An apparatus worn on a user's face comprising:
a set of goggles; and
a divider separating an air mass that touches the user's eyes and an external air mass wherein at least some portion of the divider contains a highly effective air filter.

2. The apparatus of claim 1 further comprising wherein the divider forms a substantially hermetic seal to at least one of the group comprising the skin on the user's face and the goggles.

3. The apparatus of claim 2 further comprising wherein the hermetic seal comprises at least one of the group of rubber, plastic, silicone, tape, metal fixation devices, non-metal fixation devices or other similar materials.

4. The apparatus of claim 1 further comprising wherein the goggles comprises at least one of the group of thin plastic surface(s), prescription eye glasses, and sunglasses.

5. The apparatus of claim 1 further comprising wherein the air filter comprises at least one of the group of an N95 filter, N99 filter and filter with bioactive substances.

6. The apparatus of claim 1 further comprising wherein a device is used to keep the seal firmly pressed against the skin of the face comprising at least one of the group of string, ties, rope, springs, magnets, or other similar type devices.

7. The apparatus of claim 1 further comprising wherein a one way valve is placed at a portion of the divider and wherein the filter is placed in proximity to the one way valve.

8. The apparatus of claim 1 further comprising wherein the filter can be removed and replaced.

9. The apparatus of claim 1 further comprising wherein the filter can be sterilized.

10. An apparatus adapted to be worn on a user's face for protecting the user from acquiring an airborne infection through the user's eyes comprising:
a set of goggles wherein a first portion is adapted to be sealed to the user's face around the eyes and wherein a second portion consists of an opening,
wherein the opening connects a medium of air outside the user's eyeballs to an external environment, and
wherein the opening is located above a nose cutout in the set of goggles; and
a filter component placed over the second portion of the set of goggles wherein the filter component comprises:
a filter; and
an attachment device that attaches the filter to the set of goggles,
wherein all of an air mass that travels from the external environment into the medium of air outside the user's eyeballs passes through the filter,
wherein none of the air mass that travels from the external environment into the medium of air outside the user's eyeballs passes in between the filter and the attachment device, and
wherein none of the air mass that travels from the external environment into the medium of air outside the user's eyeballs passes in between the attachment device and the set of goggles.

11. The apparatus of claim 10 further comprising wherein the divider forms a substantially hermetic seal to at least one of the group comprising the skin on the user's face and the goggles.

12. The apparatus of claim 11 further comprising wherein the hermetic seal comprises at least one of the group of rubber, plastic, silicone, tape, metal fixation devices, non-metal fixation devices or other similar materials.

13. The apparatus of claim 10 further comprising wherein a set of lenses from the set of goggles is selected from the group consisting of thin plastic surface(s), prescription eye glasses, and sunglasses.

14. The apparatus of claim 10 further comprising wherein the filter is selected from the group consisting of an N95 filter and N99 filter.

15. The apparatus of claim 10 further comprising wherein a strap is adapted to keep the set of goggles firmly pressed against the user's face to cause the first portion to be substantially hermetically sealed.

16. The apparatus of claim 10 further comprising wherein a one way valve is placed at a portion of the divider and wherein the filter is placed in proximity to the one way valve.

17. The apparatus of claim 10 further comprising wherein the filter component can be removed and replaced.

18. The apparatus of claim 10 further comprising wherein the filter component can be sterilized.

19. An apparatus worn on a subject's face comprising:
a set of goggles wherein at least one lens of the set of goggles contains at least one hole; and
an air filter placed at the sites of the at least one hole.

20. An apparatus comprising:
an air filter; and
an adhesive substance to connect the high efficiency air filter to a portion of a set of goggles.
